(19) United States
(12) Patent Application Publication
(10) Pub. No.: US 2011/0046501 A1
(43) Pub. Date: Feb. 24, 2011

(54) OPTIMIZATION OF A FLOW SENSOR

(76) Inventor: Andreas Albert, Erlangen (DE)

Correspondence Address: KREMBLAS & FOSTER, 7632 SLATE RIDGE BOULEVARD, REYNOLDSBURG, OH, 43068, US

(21) Appl. No.: 12/739,985

(22) PCT Filed: Oct. 29, 2008
(86) PCT No.: PCT/EP2008/064667

§ 371 Date: Jul. 27, 2010

(30) Foreign Application Priority Data
Oct. 31, 2007 (EP) 07119713.1

(51) Int. Cl. A61B 5/087

(52) U.S. Cl. 600/538

(57) ABSTRACT

A sensor for a flow measuring apparatus for lung function diagnostics or performance diagnostics, which sensor has an inner chamber, in particular an inner chamber which is oblong in the direction of flow, and has a resistor for producing a differential pressure between an inner chamber section located upstream from the resistor and between an inner chamber section located downstream from the resistor, which resistor consists of a planar component permeated by openings, which resistor is arranged at an angle between 20° and less than 90° to the direction of flow or to the longitudinal extension of the inner chamber.

The invention relates to a flow sensor for measuring tidal volume and flow rate in lung function diagnostics and performance diagnostics in accordance with the generic part of claim 1.

Sensors for measuring flow are used in a plurality of different embodiments and processes. Current measuring processes for flow measurement operate, for example, according to the principle of the multiple heat wire, with ultrasonic measurements or with differential pressure measuring. In order to be used in lung function diagnostics and performance diagnostics the test person breathes through a mouthpiece connected via appropriate hose connections to a flow sensor through which the test person breathes via the mouthpiece and the hose connection. In this manner the flow rate of the exhaled air can be determined and from it (die Strömungsgeschwindigkeit) the tidal volume can also be calculated using the known cross sections of the flow measuring apparatus.

The invention relates to an improvement to a flow sensor for differential pressure measuring. In differential pressure measuring a flow resistor is introduced into the flow path. The pressure drop, i.e., the difference between pressure before the resistor and pressure after the resistor can be determined by measuring the pressure before and after the resistor. This pressure drop represents a measure for the rate with which the air passes through the resistor. The relationship between flow rate and pressure drop is a function here of the form of the resistor. In the case of a fine-meshed sieve as flow resistor, for example, the flow remains laminar after passing through the sieve, i.e., no turbulence is produced and a linear ratio results between differential pressure and flow rate.

DE 43 25 789 A1 describes a flow sensor in the form of a variable aperture for use in a pipe system for measuring the lung function in lung function diagnostics or performance diagnostics. In the case of such a variable aperture the resistance changes as a function of the flow rate, so that a broader range can be covered. However, variable apertures no longer have a linear connection between pressure drop and flow rate.

It is necessary for lung function diagnostics to measure flow rates in the range of 20 ml per second to 15 l per second with an accuracy of 3%. It is necessary for this to make available the most linear relationship possible between the flow rate and the pressure resulting from it. However, other criteria must be maintained that render the production of an appropriate sensor difficult. Thus, the clearance volume of the sensor must not exceed a boundary limit in order to exclude danger to the health of the test person by the re-inhalation of used air. Therefore, the flow sensor cannot be enlarged as desired. However, the sensor resistance can also not be increased as desired in order to broaden the measuring range upward in this manner since the back pressure by the sensor on the lung also must not exceed a boundary value. On the other hand, too low a resistance has a disadvantageous influence on the linearity of the measuring.

There is therefore a need to make a sensor available that has a low resistance and at the same time good linearity with a small clearance volume.

This problem is solved with a sensor according to Claim 1. Advantageous further developments result from the subclaims.

A sensor in accordance with the invention has an inner chamber in which a resistor with a planar design is arranged against the air flow. This planar resistor has an angle of its resistance plane against the direction of flow that differs by 90 degrees. As a result of the arrangement of the resistor at an angle to the direction of flow a greater resistance surface can be used with a steady sensor volume, as a result of which the resistance and therewith the back pressure on the lung is reduced without negatively influencing the linearity of the resistance. The ratio of sensor surface of the resistor arranged at an angle to the vertical cross-sectional surface of the flow sensor, which cross-sectional surface corresponds to the surface of a conventionally arranged resistor, corresponds to a factor of

$$\frac{1}{sin α} ,$$

in which alpha is the angle of the angularly arranged resistor to the direction of flow.

The angle at which the resistor to the direction of flow is can vary in the range of 20 to under 90 degrees. Angles that are flatter than 20 degrees to the direction of flow do not bring about any significant improvement since no appreciable flow can take place any longer in the flat corners between the inner chamber and the resistor. A preferred range for the angle to the direction of flow is between 30 and 80, more preferably between 40 and 50 degrees. The resistor can be arranged especially preferably at an angle of 45 degrees.

A surface of approximately 1200 to 2400 square mm is provided for the vertical cross-sectional surface of the inner chamber for a sensor in accordance with the invention for the measuring of flow, which cross-sectional surface should preferably be in the range of 1600 to 200¹ square mm. The form of the cross section can basically be selected relatively freely and a circular or oval cross section is preferred. An elliptical cross section is especially preferably used in which the shorter main axis is between 70 and 24 mm, preferably between 19 or² 22 mm, and especially preferably 21 mm, and a longer main axis is between 22 and 32 mm, preferably between 25 and 29 mm and especially preferably 28 mm long. 1 sic2 sic

The sensor can have an angle to only one of the two main axes or to both main axes.

Further features, combinations of features, advantages and properties result from the following description of a preferred exemplary embodiment and from the drawings.

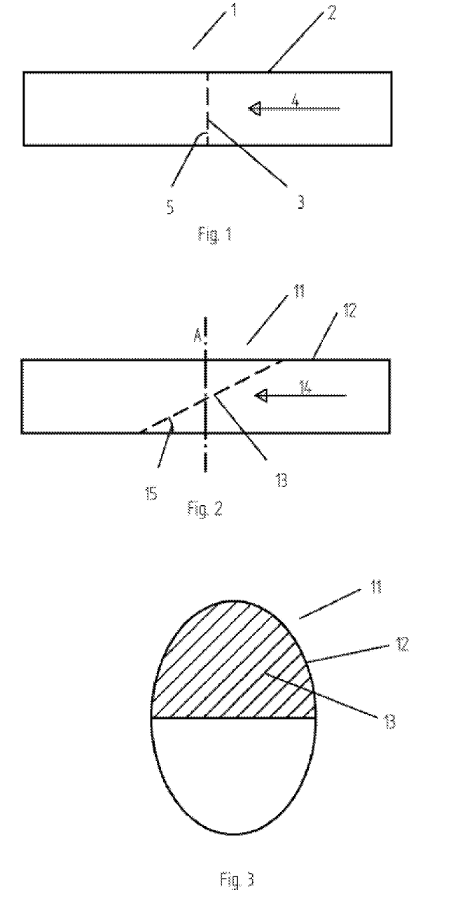

FIG. 1 shows a section through a flow sensor in accordance with the state of the art.

FIG. 2 shows a flow sensor in accordance with the invention in longitudinal section.

FIG. 3 shows a flow sensor in accordance with the invention in a cross section along line A in FIG. 2.

FIG. 1 shows a flow sensor 1 customary in the state of the art. Flow sensor 1 consists of a housing 2 designed as a hollow profile in the manner of a tube. The embodiment in FIG. 1 shows a one-part housing but multi-partite housings are just as possible. A flow resistor 3 is arranged inside the housing and is arranged vertically in the state of the art, i.e., at an angle 5 of 90 degrees to direction of flow 4. Connections for connecting the pressure measuring apparatuses (not shown in the drawings) are located in direction of flow 4 in front of and after resistor 3.

FIG. 2 shows the flow sensor 11 in accordance with the invention. Resistor 13 is arranged in the housing 12, which resistor has an angle 15 relative to direction of flow 14 that is less than 90 degrees. Even in the case of the flow sensor in accordance with the invention connections (not shown) for the connection to the pressure measuring apparatuses are present in direction of flow 14 in front of and after resistor 13 with which pressure measuring apparatuses the pressure difference and the pressure drop on the flow resistor can be measured. Housing 12 can also be constructed in one part or in multiple parts. In an especially preferred embodiment the housing is manufactured integrated with the flow resistor in the injection molding process. Depending on the requirements, a constant or a variable flow resistor can be used in this case.

FIG. 3 shows a section through the flow sensor of FIG. 2 along line A. Housing 12 has an elliptical cross section in which flow resistor 13 is arranged at an angle so that intersection line A intersects flow sensor 13 approximately in the middle.

### LIST OF REFERENCE NUMERALS

1 flow sensor
2 housing flow sensor
3 flow resistor
4 direction of flow
5 angle of flow resistor to the direction of flow
11 flow sensor
12 housing
13 flow resistor
14 direction of flow
15 angle of flow resistor to the direction of flow

1. A sensor (11) for a flow measuring apparatus for lung function diagnostics or performance diagnostics, which sensor has an inner chamber which is oblong in the direction of flow (14), and has a resistor (13) for producing a differential pressure between an inner chamber section located upstream from the resistor and an inner chamber section located downstream from the resistor, which resistor (13) consists of a planar component permeated by openings, characterized by the resistor (13) being arranged at an angle (15) between 20° and less than 90° to the direction of flow (14) or to the longitudinal extension of the inner chamber.

2. The sensor (11) according to claim 1, wherein the resistor (13) is arranged at an angle (15) between 30° and 80°.

3. The sensor (11) according to claim 2, wherein the inner chamber has a cross-sectional surface vertical to the direction of flow (14) or to a longitudinal extension of the inner chamber of 1200 to 2400 square millimeters.

4. The sensor (11) according to claim 3 wherein the inner chamber has an elliptical cross section vertical to the direction of flow (14) or the longitudinal extension.

5. The sensor (11) according to claim 4, wherein the shorter main axis of the elliptical cross section is between 17 mm and 24 mm long, and the longer main axis of the elliptical cross section is between 22 mm and 32 mm long.

6. The sensor (11) according to claim 5, wherein the shorter main axis is 21 mm long and the longer main axis is 28 mm long.

7. The sensor (11) according to claim 4 wherein the resistor 13 has an angle (15) of greater than 20° and less than 90° relative to both main axes.

8. The sensor (11) according to claim 4, wherein the resistor (13) is arranged at an angle (15) between 40° and 50° to the direction of flow.

9. The sensor (11) according to claim 8, wherein the shorter main axis of the elliptical cross section is between 19 mm and 22 mm long, and the longer main axis of the elliptical cross section is between 25 mm and 29 mm long and the inner chamber has a cross-sectional surface vertical to the direction of flow (14) or to a longitudinal extension of the inner chamber of 1600 to 2000 square millimeters.

10. The sensor (11) according to claim 9, wherein the angle (15) at which the resistor (13) is arranged is 45° to the direction of flow.
